(19) United States
(12) Patent Application Publication
(10) Pub. No.: US 2009/0095643 A1
(43) Pub. Date: Apr. 16, 2009

(54) Amperometric Method And Apparatus For Measurement Of Soft Particles In Liquids By Analyzing The Adhesion Of These Particles To An Electrode

(76) Inventors: Vesna Svetlicic, Zagreb (HR); Vera Zutic, Zagreb (HR); Amela Hozic Zimmermann, Zagreb (HR)

Correspondence Address: ST. ONGE STEWARD JOHNSTON & REENS, LLC, 986 BEDFORD STREET, STAMFORD, CT, 06905-5619, US

(21) Appl. No.: 12/253,686

(22) Filed: Oct. 17, 2008

Related U.S. Application Data
(63) Continuation of application No. PCT/HR2006/000008, filed on Apr. 19, 2006.

(51) Int. Cl. G01N 33/487

(52) U.S. Cl. 205/792; 204/403.01

(57) ABSTRACT

Method and apparatus for measurement and analysis of soft particles in liquids in the size range 1-500 μm represented by vesicles and living cells, liposomes and blood cells in particular, but also to diluted dispersions of oil droplets and other confined microparticles in liquids. The method is based on amperometric detection and analysis of single events of particle adhesion in raw samples, or after the concentration adjustment, by means of permanent record of time series of stochastic electrical signals in the real time. The detected new class of electrical signals is generated by adhesion causing the deformation, rupture and spreading of soft particles at mercury electrode in air saturated liquids. Information stored in one current pulse signal is: particle size, adhesion properties and electrode surface area occupied by the spread particle, which is characteristic for each class of particles. The current pulses, appear at irregular intervals due to the inherently stochastic nature of the I particle encounter with the electrode. The total pulse counts (N) recorded for a: fixed time interval (e.g. 100 seconds) is the measure of particle concentration; (C/particles L⁻¹) in the analyzed suspension. The pulse heights (H/μA) are proportional to the particle sizes. Distribution of pulse heights for the fixed time interval (e.g. 100 seconds) represents a relative size distribution (P). Information stored in the time series of the signals is: concentration of soft microparticles in the suspension (C) and the relative size distribution of soft microparticles (P).

### CROSS-REFERENCE TO RELATED APPLICATIONS

The present application is a continuation of pending International patent application PCT/HR2006/000008, filed Apr. 19, 2006, which designates the United States, the content of which is incorporated herein by reference.

### TECHNICAL FIELD OF THE INVENTION

The present invention relates to the method and apparatus for measurement and analysis of soft particles in liquids in the size range 1-500 μm represented by vesicles and living cells, liposomes and blood cells in particular, but also to diluted dispersions of oil droplets and other confined microparticles in liquids. Generally, according to the present invention, the term soft particles refers to organic micro-droplets, vesicles and living cells with liquid or flexible outer membrane that can readily form adhesive contact with the substrate with little or no resistance to oppose deformation. The method is based on amperometric detection and analysis of single events of particle adhesion in raw samples by means of permanent record of time series of stochastic electrical signals in the real time.

### TECHNICAL PROBLEM

The object of the present invention is to solve, in a direct, rapid and simple way, the problem of detection and characterization of soft microparticles in liquids, in particular liposomes and blood cells suspended in physiological and other electrolyte solution. The problem of detection and characterization of soft particles is relevant to monitoring quality of natural waters, technological processes in pharmaceutical and food industries and clinical research-biotechnology. The method disclosed here is simple, reliable and selectively detects individual particles regardless of the presence of solid particles.

### BACKGROUND ART

The need to develop highly sensitive instrumentation and methodologies for selective detection and characterization of soft particles in liquids in the domains of biological, biomedical, environmental, waste waters, effluents, agrochemical and food specialties control, clearly appears throughout the increasing number of publications and patents dedicated to these subjects.

Electroanalytical techniques, amperometry in particular, provide an important and convenient variety of tools for analysis. Their efficiencies, their sensitivities, and the easy way they are operated by non-specialists and also their relative low cost, make these techniques more attractive than most of physical methods for which a high level of expertise is required. Moreover, the physical dimensions and low energy consumption is equally attractive.

The most common principle for particle counting and sizing is electrochemical principle known as the <<aperture impedance>> or the <<Coulter>> principle (U.S. Pat. No. 2,656,508). According to the Coulter principle, particles themselves do not interact with the electrode surface and do not change electrode properties in the course of measurement. The measurement is conducted in such a way that particles suspended in a weak electrolyte solution are drawn through a small aperture separating two electrodes between which an electric current flows. The voltage applied across the aperture creates a “sensing zone”. As each particle passes through the aperture (or “sensing zone”) it displaces its own volume of conducting liquid, momentarily increasing the impedance of the aperture. This change in impedance produces a tiny but proportional current flow into an amplifier that converts the current fluctuation into a voltage pulse. The Coulter Principle states that amplitude of this pulse is directly proportional to the volume of the particle that produced it. Scaling these pulse heights in volume units enables a size distribution to be acquired and displayed. In addition, if a metering device is used to draw a known volume of the particle suspension through the aperture, a count of the number of pulses will yield the concentration of particles in the sample. There are several experimental methods by which one can infer information about effective interaction (adhesion) between two membranes or between a membrane and another surface which are separated by a thin liquid film. These methods include X-ray diffraction on oriented multi-layers, the surface force apparatus, micropipette aspiration and video microscopy of diluted systems (Lipowsky and Sackmann, 1995).

Adhesion of red blood cells (RBC) to solid electrodes has been demonstrated by classical paper of Gingel et al., 1975 on lead electrode and later by Goldin and Caprani, 1997 at platinum microelectrodes. However, there was neither attempt nor indication that such an approach to cell-electrode interaction could be used for cell counting or sizing.

### BACKGROUND OF THE INVENTION

According to the modified Young-Dupre equation (Adamson, 1982, Israelachvili, 1992), the total Gibbs energy of interaction between a droplet and the aqueous mercury interface is:

−ΔG=A(γ₁₂−γ₁₃−γ₂₃) (1)

where γ₁₂, γ₁₃ and γ₂₃ are surface tensions at mercury/water, mercury/non-polar organic liquid and non-polar organic liquid/water interfaces, respectively. The expression in parenthesis is equal to the spreading coefficient:

S=γ₁₂−γ₁₃−γ₂₃ (2)

For positive values of S the organic droplet will spread spontaneously and displace ions and water molecules from the interface. For S<0 the spreading process will not proceed spontaneously.

In a 0.1 M NaCl supporting electrolyte (FIG. 1), the surface tension vary at least 100 mJ/m² in the potential window of 2 V, since:

$${γ}_{12} = {γ}_{12}^{0} - {\int }_{0}^{{E}_{\pm }^{′}} {σ}_{Hg} d {E}_{\pm }^{′} \qquad ( 3 )$$

γ₁₂⁰ is surface tension at the potential of zero charge of the electrode—E_{pzc} and E′=E−E_{pzc}.

If γ₁₃ is independent of the applied potential, the spreading coefficient S and thus the adhesion of droplets from dispersion will be controlled by γ₁₂ only. This was confirmed by wetting behavior of hydrocarbon droplets at the mercury electrode/aqueous electrolyte interface (Ivosevic et al., 1994). Critical surface tension of wetting determined from critical potentials at the dropping mercury electrode (DME) was equal to the prediction based on the Good-Girifalco-Fowkes equation (Fowkes, 1963) for the mercury/water/hexadecane system (418.26 mJ/m²). These experimental findings prove unambiguously that adhesion and spreading of hydrocarbon droplets at mercury electrode result in direct contact between mercury and hydrocarbon, i.e. the adhesion in proper physical-chemical sense.

Hexadecane Droplet at Mercury Pool Electrode/Aqueous Electrolyte Interface

Hexadecane is the highest n-alkane that is liquid at room temperature with a large number of interfacial data available for calculations of wetting equilibrium. The effects of electrical potential on the shape of hexadecane droplet at mercury pool/aqueous electrolyte interface are shown in FIGS. 2A and 2B. Hexadecane drop (volume 70 μl) forms a planar convex lens at the constant potential of −550 mV (Epzc). The lens changes its shape to semispherical with shifting the potential to the more negative values. The droplet remains firmly attached to the mercury interface even at the potentials more negative than the critical potential of wetting, Ec—=−730 mV. With further changing of potential towards −1400 mV, the droplet changes the shape from semispherical to an ideal sphere, while the contact area decreases. FIG. 2B shows contours of droplet shapes at different constant potentials. Only at more negative over-potential, −1456 mV, hexadecane droplet detaches and rises to the surface by buoyancy. At the given potentials, the shapes of hexadecane droplet were established instantaneously. The particular shapes were reproducible in independent experimental series and perfectly repeatable by the successive changes of potential within the range where detachment does not take place. The difference between the potential of the detachment of hexadecane droplet and the critical potential of wetting of ΔE=−700 mV, corresponds to the difference in surface energy of 80 mJ/m2. This difference in surface energy can be interpreted by the presence of an underlying hexadecane monolayer, formed immediately upon droplet deposition at the mercury electrode/aqueous electrolyte interface. As little as 0.014% of 70 μL droplet volume would be sufficient to form a monolayer over the entire interface (assuming that the surface area per molecule is 20 Å²).

Electrochemical Sensing of Soft Particles at the Mercury Drop Electrode

Organic microdroplets, vesicles and cells with liquid or flexible outer membrane can readily form adhesive contact with the substrate with little or no resistance to oppose deformation. Attractive interaction between a soft particle and the mercury drop electrode results in a double layer charge displacement in the form of adhesion signal schematically shown in FIG. 3. The charge displacement causes a flow of compensating current, ID that is directly related to the formation of adhesion contact, which in turn depends on the particle deformability and the particle size:

$${I}_{D} = - \frac{d A}{d t} {σ}_{E} \qquad ( 4 )$$

A is the area of the contact interface, t is time and σ_{E} is the surface charge density of the electrode/aqueous electrolyte interface. The amount of displaced charge, q_{D} is determined from the adhesion signal (FIG. 3) by integrating current over time:

$${q}_{D} = {\int }_{{t}_{1}}^{{t}_{1} + τ} i d t \qquad ( 5 )$$

The area of the contact interface formed, Ac, can then be determined with precision, surface charge of the electrode being known:

$${A}_{C} = \frac{{q}_{D}}{{σ}_{E}} \qquad ( 6 )$$

### DISCLOSURE OF THE INVENTION

The invention relates to the electrochemical sensing of soft particles in liquids using electroanalytical technique of chronoamperometry or chronopotentiometry. In contrast to the existing electrochemical techniques for particle counting and size determination in liquids, the subject of this invention is the method based on particle adhesion upon impact with the electrode at the predetermined constant potential or constant current.

Adhesion with the liquid electrode is the basic principle for an electrochemical imaging of individual microparticles. Based on the adhesion principle, the detection of soft particles in liquids becomes simple and reliable. Moreover, presence of solid particles does not interfere with the measurement.

Electrochemical imaging of individual particles is performed in raw samples or after the concentration adjustment. The mercury drop electrode has renewable atomically smooth surface, electrically chargeable in the broad potential range. The surface tension and surface charge at the mercury/electrolyte solutions interface can be controlled by the externally applied potential or current.

### BRIEF DESCRIPTION OF THE DRAWINGS

FIG. 1 shows potential control of mercury electrode surface tension (γ₁₂) and surface charge (σ₁) in aqueous electrolyte solution (0.1 M NaCl);

FIG. 2(A) shows photographs of n-hexadecane droplet at mercury pool/aqueous electrolyte interface taken at the constant potentials of −550 mV and −1400 mV, bar denotes 2 mm;

FIG. 2(B) shows contours of droplet shapes at the potentials: −550 mV (1), −1300 mV (2), −1400 mV (3) and −1450 mV (4);

FIG. 3 shows schematically presentation of the amperometric adhesion signal;

FIG. 4 shows adhesion signal in the form of current pulse without oxygen and amplified by oxygen reduction—Example: erythrocyte cell in PBS at −400 mV;

FIG. 5a shows adhesion signal in the form of current pulse;

FIG. 5b shows adhesion signals in the form of current pulse in air saturated suspension of lymphocytes in PBS at −400 mV;

FIGS. 6a, 6b show the reference calibration curves: pulse counts over 100 seconds plotted against cell density in suspensions of Dunaliella tertiolecta cells in the two different media: (a) natural seawater and (b) 0.1 M NaCl;

FIG. 7 shows adhesion signals of liposomes in the form of current pulse, recorded at two predetermined potentials: (a) −400 mV and (b) −800 mV. Each pulse corresponds to one liposome adhesion at the mercury drop. Framed signals are shown at extended time scale;

FIG. 8 shows schematic presentation of the apparatus for counting and sizing soft microparticles in liquids;

FIG. 9. shows 4000 ms segment of the 100 seconds stored time series for red blood cells (RBC) analysis as it appears in the Analytical Screen using command “compare” displaying simultaneously the original and the extracted data sets;

FIG. 10. shows 4000 ms segment of the 100 seconds stored time series for liposome analysis as it appears in the Analytical Screen using command “compare” displaying simultaneously the original and the extracted data sets.

### DETAILED DESCRIPTION OF THE INVENTION

Adhesion is a principal surface interaction leading to particle aggregation and basic principle of invention: electrochemical method of counting and measuring soft microparticles in their natural environments. Individual particles (blood cells and liposomes) are counted and measured through the following sequence of events at the mercury electrode: (1) adhesion, (2) deformation, (3) rupture and spreading, resulting in specific signals for different particle classes.

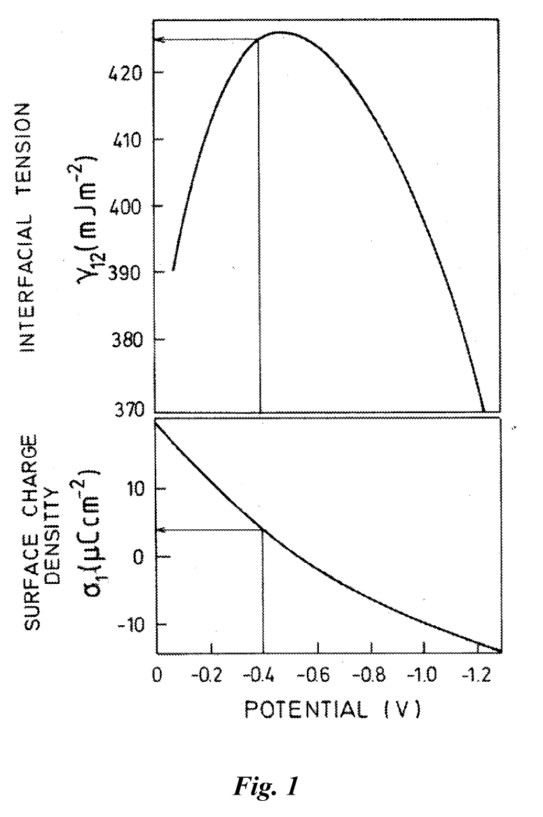

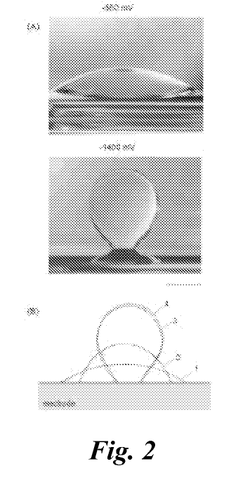

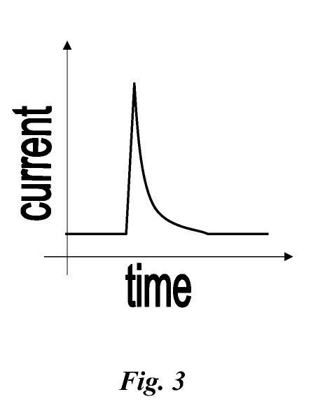

Mercury electrode as a substrate for adhesion studies has several advantages: it is atomically smooth, chemically inert and possesses high surface energy. Mercury drop electrode is of any construction with periodical dislodging of Is to 10 s. By varying the electrical potential from 0 to −1.2 V, the surface tension is precisely controlled in the range of 60 mJ/m² and surface charge density from +18 to −12 μC/cm² (FIG. 1). The main advantage of mercury drop electrode in particle analysis is its renewable surface, which is isotropic with the respect to its physicochemical properties. The reproducible formation of a clean electrode surface is exploited for collection of a large set of data under identical experimental conditions. Electrode surface area renews with constant frequency (2 s described herein), which is crucial for a useful signal extraction.

The mercury drop electrode is immersed in particle suspension and set at a constant predetermined potential where the strong attraction force acts between the mercury surface and the particles. Any potential within the range of −100 mV to −1100 mV can be chosen for the analysis. The most commonly applied potential of −40 OmV is marked in FIG. 1, together with the corresponding values of interfacial tension and surface charge.

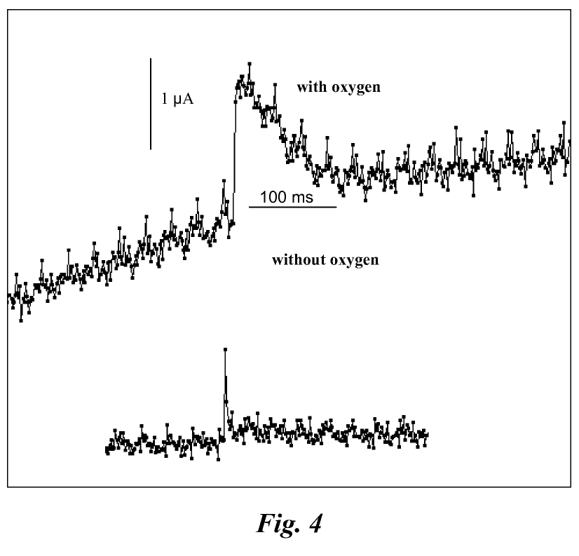

Different classes of particles produce distinctive adhesion signals in the form of current pulses upon impact with the electrode at a constant predetermined potential. We took advantage of a naturally occurring reagent in the particle suspension—dissolved molecular oxygen—and its charge transfer reaction at the mercury electrode, to amplify the current pulses produced by adhesion events of soft particles. The amplifying effect of the charge transfer reaction of oxygen reduction on the pulse magnitude and duration is illustrated in FIG. 4. Impact of a soft particle with the mercury surface, exemplified by erythrocyte in FIG. 4 is registered as a well resolved current pulse on ms time scale. The current pulse is composed of: a) flow of current caused by ion displacement from the contact interface of the cell with the mercury electrode (eq. 4), and b) additional oxygen reduction current caused by transient increase of oxygen supply through turbulence at the 3-phase contact boundary (solution/cell/mercury electrode) caused by surface tension gradient.

Process leading to the flow of current I_{D} (eq. 4) is the surface process. Additional oxygen reduction is the volume reaction triggered by the surface process and lasts until the turbulent streaming attenuates. Currents caused by the two processes are cumulative. The pulse duration is increased by an order of magnitude and so is the integral of current over time. This amplification enables detection and recording of current pulses.

The detected new class of electrical signals is generated by adhesion causing the deformation, rupture and spreading of soft particles at mercury electrode in air saturated liquids. Blood cells and liposomes conveniently suspended in phosphate buffer saline (PBS) produce measurable current pulses. Each current pulse is the result of impact of a soft particle with mercury electrode. The current pulse is characterized by maximum height, duration and by the decrease bellow base line current, where the base line current is the current time curve of oxygen reduction in absence of particles. The pulse shape is asymmetrical with the steep rise to the maximum height and with a slower descending part. The pulse height (10 nA-100 μA) is proportional to the particle size. The descending profile is determined by the rate of spreading. The rate of spreading is related to the spreading coefficient S (eq. T), which is characteristic of a given particle class.

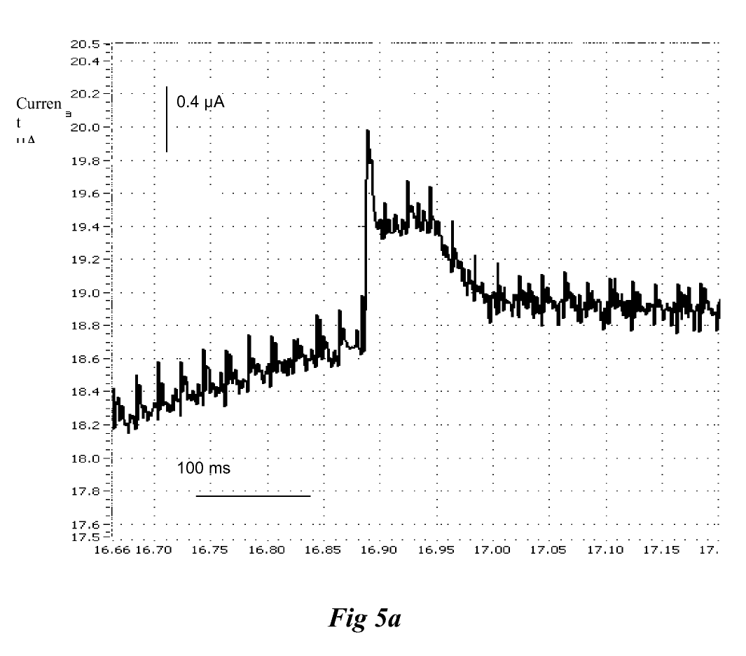

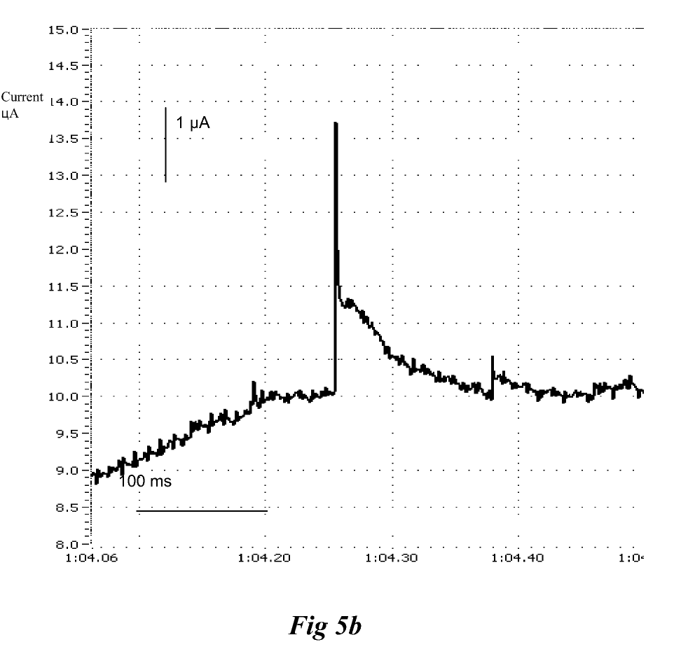

The decrease bellow the base line current corresponds to the surface area of the electrode occupied by the spread particle. In summary, information stored in one current pulse signal are: particle size, adhesion properties and electrode surface area occupied by the spread particle which is characteristic for each class of particles as exemplified in FIGS. 5a and 5b for erythrocyte and lymphocyte single cell.

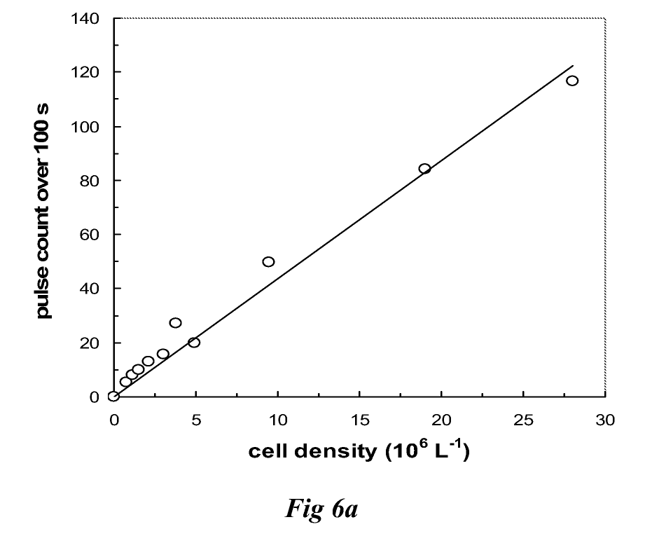

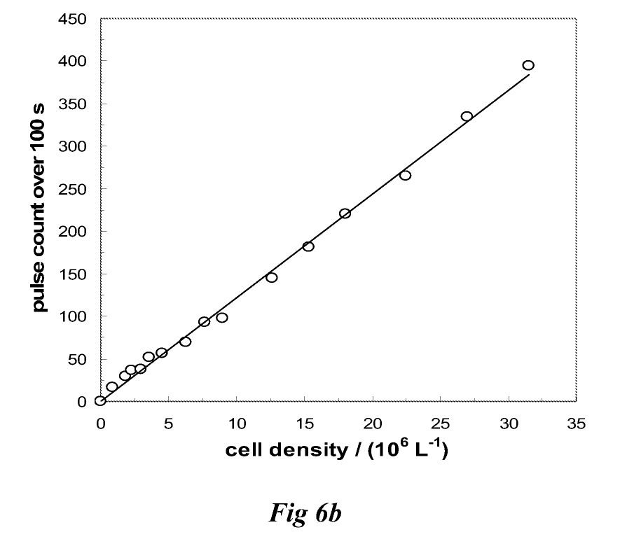

The current pulses (10 nA to 100 μA, duration in the range of 10 ms to 100 ms) appear at irregular intervals due to the inherently stochastic nature of the particle encounter with the electrode. The total pulse counts (N) recorded for a fixed time interval (e.g. 100 seconds) is the measure of particle concentration (C/particles L⁻¹) in the analyzed suspension. The pulse heights (H/μA) are proportional to the particle sizes. Distribution of pulse heights for the fixed time interval (e.g. 100 seconds) represents a relative size distribution (P). In summary, information stored in the time series of the signals is: concentration of soft microparticles in the suspension (C) and the relative size distribution of soft microparticles (P). To acquire particle concentration and particle size distribution in the particular sample a normalization of current pulse frequency and current pulse heights is needed. This is achieved using a referent calibration curve. Collecting pulse-counts over 100 s in particle suspensions of varying densities is used to construct the referent calibration curves exemplified in FIGS. 6a and 6b. It is recommended to construct the reference calibration curve for each particle class in respective medium. For samples of unknown particles the convenient reference particle system is Dunaliella tertiolecta cell suspension. Dunaliella tertiolecta cells are suitable reference particle system because of their uniform size (6-10 μm), detection in a broad potential range (from −100 mV to −1100 mV) and euryhaline nature (supports wide salinity range). The cells are simple to grow in the laboratory. In a variety of aqueous electrolyte solutions (FIGS. 6a and 6b) they form stable suspensions of single cells.

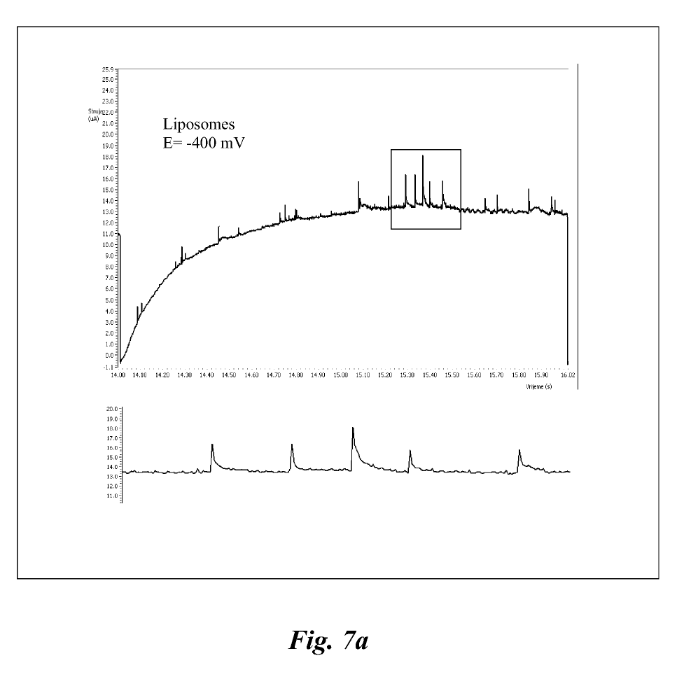

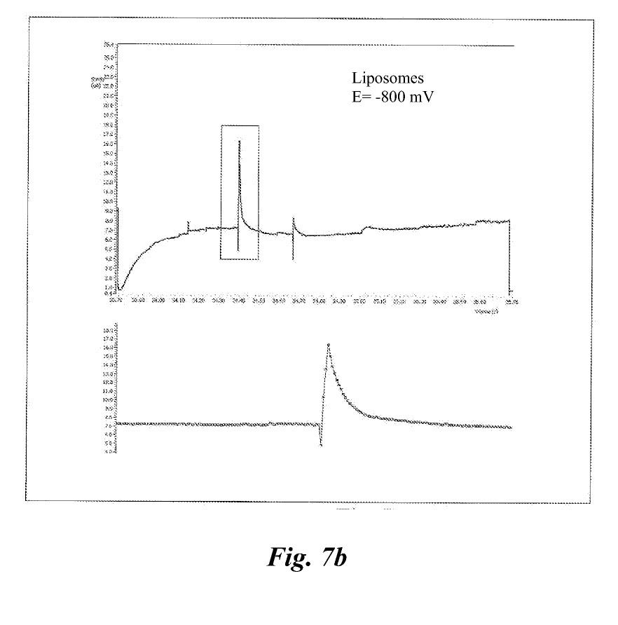

The shape of current pulses at the predetermined potential is characteristic for the given particle class. This fact is exemplified by current pulses of an erythrocyte (FIG. 5a) and a lymphocyte (FIG. 5b) recorded at the predetermined potential of −400 mV. The additional characterization within the same particle class is achieved in a very simple manner, by selecting a number of different predetermined potentials. This is exemplified by the current pulses recorded in a liposome suspension at −400 mV (FIG. 7a) and −800 mV (FIG. 7b).

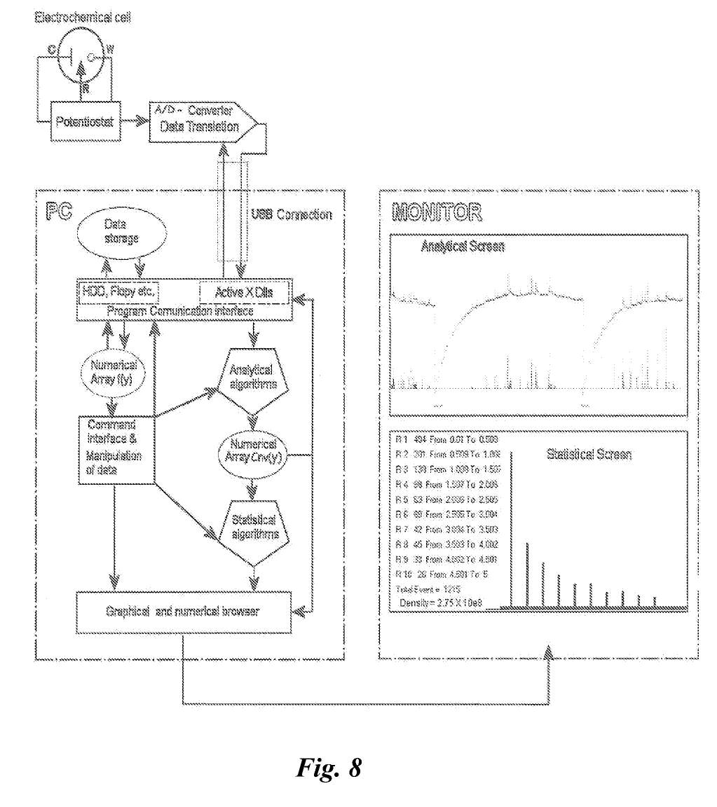

An embodiment of the present invention will be described below with the reference to FIG. 8.

Apparatus for measuring and analysis of soft microparticles in liquids in the size range 1-500 μm is described. The following soft particles can be detected and analyzed: vesicles and living cells, liposomes and blood cells in particular, but also oil droplets and other confined soft microparticles. Apparatus (FIG. 8) consists of:

1. sensor body comprising electrochemical cell with two or more electrodes and potentiostat or galvanostat;

2. electronic module comprising A/D converter and system for data translation;

3. signal processing unit (PC) comprising monitor with signal display in running mode and display of results.

According to the embodiment of present invention, sensor body comprises electrochemical cell with three-electrode configuration and the standard potentiostat. The working electrode is mercury drop electrode of any construction with a periodical dislodging in time intervals of Is to 10 s. Counter electrode and reference electrode are of any kind and are chosen according to the volume of sample and size of working electrode. The electronic module is used for analogous data acquisition, data translation and communication with signal processing unit. The DAC UCB input device enables communication with signal processing unit. Signal processing unit enables recognition, recording and storing stochastic electrical signals in the range of the time intervals and time resolution of data points suitable for the quantification and the real time display. This is achieved using the original application developed in visual C⁺⁺ for OS MS Windows 98, 2000 and XP. Electronic module communicates with the signal processing unit through USB connection.

The application of operational segment is directed toward a selective extraction and counting of signals (pulses) corresponding to real adhesion events of diverse microparticle classes with the possibility for a direct identification of signals of each individual particle through the analytical-statistical interface.

Internal architecture of the program, shown on FIG. 8, includes the communication with all relevant sources of data received from the sensor body. Loading of data into the Numerical Array f(y) is proceeded through the command interface, either from the computer hard disc or any other data file system, as a bit stream from the A/D Converter and the system for data translation in real time. The communication interface ensures the input of selected data of the Numerical Array f(y) and Numerical Array Oav(y) into the data structure of the computer. The communication interface also controls the A/D Converter by means of API functions in Windows system. Conditions for further data processing are achieved by loading the data from any of sources into the Numerical Array f(y). The data are then stored into the data structure of the computer or directed for further processing using the analytical algorithms developed. Specification of parameters for processing and their execution is achieved through the set of procedures within the Command Interface and Manipulation of Data. These procedures also run the procedure for statistical analysis algorithm. By means of statistical algorithms the program determines the distribution of particle adhesion events at the dynamic electrode surface and sorts the data according to their amplitudes. All obtained results and monitoring of the input of current sequence from the sensor body are shown by means of the graphical and numerical browser in the form of parallel graphic presentations of the time series (e.g. 100 seconds) and subsequently processed data series in the form of single pulse amplitude displayed in the Analytical Screen. Statistical Screen displays the histogram and the numerical data about the distribution of pulse amplitudes. Additional program module “Global Properties” enables control and selection of parameters for detection, statistical distribution of pulse amplitudes and statistical calibration of the system for interpretation of number of detected events in terms of particle concentration in suspension. Particle concentration (C, particles L⁻¹) is evaluated from the set of data adjusted to the empirical data comprising the referent calibration curve. Each input of data contributes to the database and corresponding calibration function is activated through the selection of a unique dataset by the algorithm for synthesis of calibration curve. Such calibration function interrelates with group of measured data concerning the total pulse count (N) and interprets them as particle concentration (C). The information on the total pulse count (N) and particle concentration (C) is displayed at the bottom of the Analytical Screen.

Statistical algorithms evaluate relative particle size distribution. Statistical algorithms contains amplitude filter with 10 amplitude ranges and fields for input of minimum and maximum value of a range. Data are displayed in statistical screen as numerical and graphical presentation.

Besides particle concentration and their size distribution the additional information can be extracted from the unique shape of the current pulse that results from a single particle adhesion with the mercury drop electrode. The internal program architecture allows implementation of additional criteria for selection and characterization of single particles, such as the integration of pulse current over time or its shape (pattern recognition).

Application of program enables:

1. signal display with adjustable time resolution (lower limit 0.1 ms) 2 j,

2. storage of long time series (IGB) and analysis of stochastic events in terms of signal frequency and size distribution

3 specified analysis of individual signals with signal amplitude in the range from 10 nA to 100 uA.

Signals are displayed in real time (running mode) and the analyzed data in <<AnalyticalScreen>> and <<Statistical Screen>>. Extracting algorithms properties for signal analyses are selected from the screen <<Global Properties)).

Requirements for Optimal Measurement Performance
Mercury drop electrode with growing surface and periodical dislodging of 2 seconds
Ionic strength of particle suspension should be in the range of 0.01 to 1 M. Preferred ionic strength is that of physiological solution, e.g. PBS. Other examples of used media are: seawater, brackish waters, freshwaters and wastewaters.
Optimum concentration range of particles in suspension for direct measurement is 5×10⁵ to 2×10⁸ particles/L.
The particle suspension should be equilibrated with the air at ambient condition.
The measurement should be carried out at known temperature.
Operating temperature 10-40° C.

Special Features:
The method and apparatus are suitable for counting and sizing of monomodal and polymodal suspensions.
No pretreatment is needed such as filtration, centrifugation and separation.
Presence of solid particles does not interfere with the measurement.
Measured parameters are independent of sample volume.

To acquire particle concentration and particle size distribution in the particular sample the scaling of current pulse frequency and current pulse heights is needed. This is achieved by the calibration curve using model particles in the same medium.

Frequency of signals is commonly translated into particle concentration using a calibration curve with Dunaliella tertiolecta cells as standard particles.

### EXAMPLE 1

### Monomodal Distribution of Particles (“Human RBC)

The diluted suspension of human RBC in PBS is placed in the electrochemical cell with the three-electrode system properly connected to the potentiostat (scheme FIG. 8). Electrochemical cell can be any glass vessel no particular shape requirement. The vessel should support the minimum of 1 ml of liquid sample and allow immersion of working electrode, counter electrode and reference electrode. Working electrode is a dropping mercury electrode with the drop life 2.0 seconds, flow rate 6 mg/s and maximum surface area 4.57 mm². Counter electrode is a coiled platinum wire, preferably 1 mm in diameter. Reference electrode is Ag/AgCl (0.1 M NaCl) which is separated from the analyzed particle suspension by a ceramic frit. The potentiostat has power supply and the main electronic board including potentiostatic circuits, the current to voltage converters and corresponding switching devices.

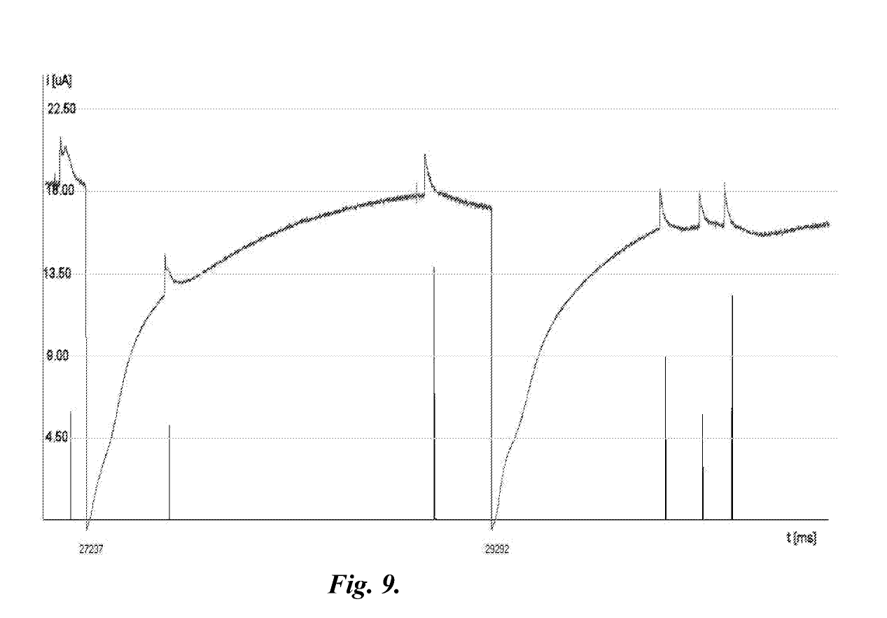

The electrochemical cell is open to air and temperature set at 20° C. The constant potential of −400 mV is applied. The data collection time is set at 100 s (50 mercury drops). The current-time curves are displayed using electronic module and signal processing unit (scheme FIG. 8). The display and manipulation of stored data are enabled through the graphical command screen in OS MS Windows. Selection of algorithm parameters to be applied in the analysis of the original imported data leads to an instant execution over the entire captured data series and generation of executed data. FIG. 9 illustrates data as they appear in the Analytical Screen using command “compare” that enables a simultaneous display of the originally stored data series and the extracted data series. Out of 100,000 data points, FIG. 9 presents data points from the point 27,037 to the point 31,037, where 6 pulses appear. Each pulse is the result of impact of one RBC with mercury electrode. Number of total pulses in time series (100 s) is 110. For the entire series the pulse heights are in the range of 1.95 μA to 2.50 μA and duration between 80 ms to 100 ms. The extracted data series (current pulses) free of electrical noise and the background current is processed statistically over selectively determined categories yielding particle concentration and size distribution. In this way, total number of pulse counts (110 in this particular example) is translated into concentration of 7×10⁶ RBC L⁻¹, from the referent calibration curve that stands in the relation with the data set for total pulse counts. The resulting concentration is confirmed using optical microscopy.

### EXAMPLE 2

### Polymodal Distribution of Particles (Liposomes)

The diluted liposome suspension in PBS (pH 7.47) is placed in the electrochemical cell with the three electrode system properly connected to the potentiostat (scheme FIG. 8). Electrochemical cell can be any glass vessel no particular shape requirement. The vessel should support the minimum of 1 ml of liquid sample and allow immersion of working electrode, counter electrode and reference electrode. Working electrode is a dropping mercury electrode with the drop life 2.0 seconds, flow rate 6 mg/s and maximum surface area 4.57 mm². Counter electrode is a coiled platinum wire, preferably 1 mm in diameter. Reference electrode is Ag/AgCl (0.1 M NaCl) which is separated from the analyzed particle suspension by a ceramic frit. The potentiostat has power supply and the main electronic board including potentiostatic circuits, the current to voltage converters and corresponding switching devices.

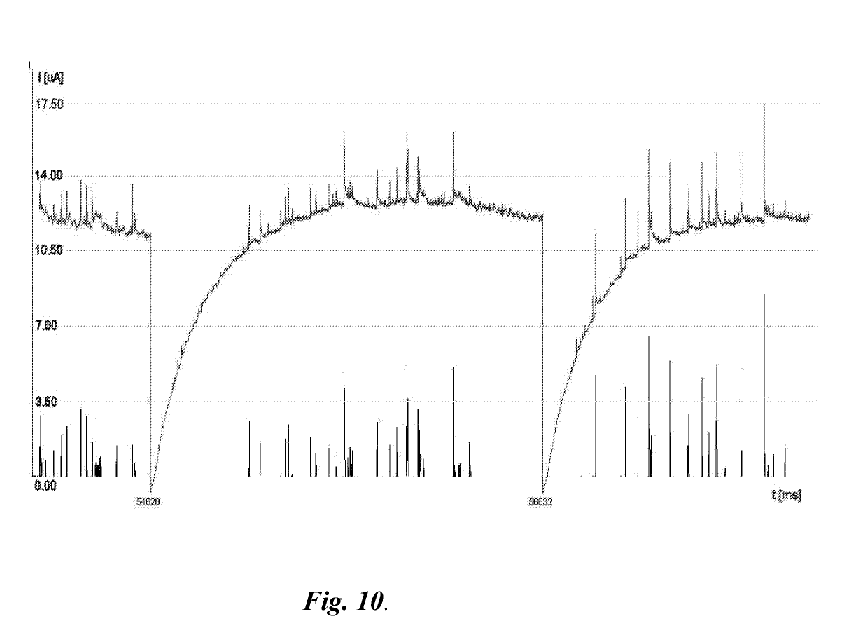

The electrochemical cell is open to air and temperature set at 20° C. The constant potential of −400 mV is applied. The data collection time is set at 100 s (50 mercury drops). The current-time curves are displayed using electronic module and signal processing unit (scheme FIG. 8). The display and manipulation of stored data are enabled through the graphical command screen in OS MS Windows. Selection of algorithm parameters to be applied in the analysis of the original imported data leads to an instant execution over the entire captured data series and generation of executed data. FIG. 10 illustrates data as they appear in the Analytical Screen using command “compare” that enables a simultaneous display of the originally stored data series and the extracted data series. Out of 100,000 data points, FIG. 10 presents data points from the point 54,058 to the point 58,058, where 46 pulses appear. Each pulse is the result of impact of one liposome with mercury electrode. Number of pulses in time series (100 s) is 1215. For the entire series the pulse heights are in the range of 0.2 μA to 32.5 μA and duration between 2 ms to 300 ms. The extracted data series (current pulses) free of electrical noise and the background current is processed statistically over selectively determined categories yielding particle concentration and size distribution. In this way, total number of pulse counts (1215 in this particular example) is translated into concentration of 2.75×10⁸ liposomes L⁻¹, from the referent calibration curve (reference particle system is Dunaliella tertiolecta cell suspension in PBS) that stands in the relation with the data set for total pulse counts. Liposome stock suspension (10 g/L) in PBS, pH 7.47 was prepared by thin-film hydration method (G. Gregoriadis, 1976). Phosphatidil choline, cholesterol, and dihexadecyl phosphate were dissolved in chlorophorm in the molar ration 7:5:1.

Apparatus for measuring and analysis and method for measurement of soft particles is used for monitoring quality of natural waters, technological processes in pharmaceutical and food industries and clinical research-biotechnology. In particular:
Monitoring stability/aggregation in soft particle suspensions of importance for food, cosmetics and personal care industries, oil industry and waste waters treatments
In pharmaceutical industry for quality control and characterization of vesicles or other flexible structures where active products are imbedded (drug or vaccine delivery)
Clinical studies for monitoring blood cells in health and disease states before, during and after the therapy
Clinical studies for monitoring blood cells in health and disease states before, during and after the therapy
Direct measurement of the surface charge density for living cells with and without autonomous movements (e.g. lymphocytes and spermatozoids) difficult to achieve by electrophoresis
Electrochemical imaging of soft microparticles—pattern recognition
Innovative tool in electroanalysis (sophisticated signal analyzer and software for amperometric stochastic signals)

1. Apparatus for measurement and analysis of vesicles and living cells, in particular liposomes and blood cells, in the size range 1-500 μm in liquids comprising:
sensor body comprising the electrochemical cell with system of two or more electrodes configuration whereby working electrode is Hg-drop electrode wherein said electrode is of any construction with periodical dislodging if 1 s to 10 s, and potentiostat or galvanostat for amperometric or potentiometric detection in a times series,
electronic module for analogous data acquisition, data translation and communication with signal processing unit, and
signal processing unit for analysis of the current pulses e.g. pulse height, distribution of pulses for a fixed time and total number of pulses per fixed time comprising program communication interface, data storage system, command interface and manipulation of data unit, graphical and numerical browser with analytical and statistical screen.

2. Apparatus according to claim 1 whereby electrochemical cell can be any glass vessel no particular shape requirement, the said vessel supports the minimum of 1 ml of liquid sample in order to allow immersion of working electrode, counter electrode and reference electrode.

3. Apparatus according to claim 1 whereby counter electrode and reference electrode are of any kind wherein said counter and reference electrode are chosen according to the volume of sample and size of working electrode.

4. Apparatus according to claim 1 whereby electronic module comprising A/D converter and system for data translation, the said electronic module communicates with signal processing unit though USB connection.

5. Apparatus according to claim 1 whereby signal processing unit performs recognition, recording and storing stochastic electrical signals in the range of the time intervals and time resolution of data points suitable for the quantification and the real time display.

6. Apparatus according to claim 1 whereby through analytical screen monitoring of input of current sequence is performed by means of graphical and numerical browser and parallel graphic presentations of the times series and subsequently processed data series in the form of single pulse amplitudes.

7. Apparatus according to claim 1 whereby though statistical screen monitoring of histogram and numerical data about distribution of pulse amplitudes is performed.

8. Apparatus according to claim 1 whereby group of procedures by command interface and manipulation of data unit performs and operates with process specification parameters, analytical and statistical analysis algorithms.

9. Method for measurement and analysis of vesicles and living cells, in particular liposomes and blood cells, in the size range 1-500 μm in liquids comprising following steps:
filling the electrochemical cell with any volume of analyzed suspension that would allow submersion of electrodes, and equilibration of particle suspension with air at ambient condition at the operating temperature,
applying constant predetermined potential or current,
monitoring of preliminary current-time or potential-time series and adjustment of sample concentration if necessary,
inputting of data to database from which by selection of a unique data set by algorithm for synthesis of calibration curve, the corresponding calibration function is activated,
normalization of current pulse frequency and current pulse heights using referent calibration curve,
loading of data into numerical field f(y) through command interface, from the computer hard disc or any other data file system as a bit stream from A/D converter and system for data translation in real time,
storing of data into data structure of the computer for further data processing by using analytical algorithms,
determination of distribution of particle adhesion events at dynamic electrode surface by means of statistical algorithms program and sorting data according to their amplitudes,
monitoring of input/output of data sequence by means of graphical and numerical browser in the form of parallel graphic presentations of the time series and subsequently processed data series in the form of single pulse amplitude displayed in analytical screen,
control of selected parameters for detection and extraction of detected events, statistical distribution of pulse amplitudes and statistical calibration of the system for interpretation of number of detected events in terms of particle concentration in suspension,
recognition, recording and storing stochastic amperometric or potentiometric signals of current—time curves or potential—time curves using electronic module and signal processing unit,
displaying and manipulation of data through the command and communication interface,
signal display with adjustable time resolution,
storing of long time series and analysis of stochastic events in terms of signal frequency and size distribution, and
analyzing of individual signals in current time series with signal amplitude in the range from 10 nA to 100 μA wherein the pulse height is proportional to the particle size, distribution of pulse heights for fixed time interval represents a relative size distribution and total pulse counts (N) recorded for a fixed time interval is particle concentration C/particles L⁻¹.

10. Method according to claim 9 whereby unique shape of the current pulse results from a single particle adhesion with the mercury drop electrode wherein the shape of current pulses at the predetermined potential is characteristic for the given particle class.

11. Method according to claim 9 whereby operating temperature is in the range between 10 to 40° C.

12. Method according to claim 9 whereby adjusting the concentration in the range of 5×10⁵ to 3×10⁸ particles per L using suspension medium.

13. Method according to claim 9 whereby applied potential depends on the kind of reference electrode.

14. Method according to claim 9 whereby applied potential is in range of −100 to −1100 mV against Ag/AgCl reference electrode.

15. Method according to claim 9 whereby applied potential is −400 mV.

16. Method according to claim 9 whereby presence of dissolved molecular oxygen is used for pulse amplification.

17. Method according to claim 9 whereby for samples of unknown particles the reference particle system is Dunaliella tertiolecta cell suspension.

18. Method according to claim 9 whereby particle concentration (C/particles L⁻¹) is evaluated from the set of data adjusted to the empirical data comprising the referent calibration curve wherein calibration function interrelates with group of measured data concerning the total pulse count (N) and interprets them as particle concentration (C).

19. Method according to claim 9 whereby statistical algorithms evaluate relative particle size distribution.

20. Method according to claim 9 whereby statistical algorithms contains amplitude filter with 10 amplitude ranges and fields for input of minimum and maximum value of a range.

21. Method according to claim 9 whereby the internal program architecture allows implementation of additional criteria for selection and characterization of single particles, such as the integration of pulse current over time or its shape.

22. Use of method and apparatus for measurement and analysis of vesicles and living cells, in particular liposomes and blood cells, in the size range 1-500 μm in liquids in clinical researches and biotechnology for monitoring blood cells in health and disease states before, during and after the therapy.
